(19) United States
(12) Patent Application Publication
(10) Pub. No.: US 2011/0177046 A1
(43) Pub. Date: Jul. 21, 2011

(54) DITHIAZOLIDINE AND THIAZOLIDINE DERIVATIVES AS ANTICANCER AGENTS

(75) Inventors: Björn Eriksson, Umea (SE); Jocob Westman, Jalasa (SE)

(73) Assignee: Betagenon AB

(21) Appl. No.: 12/671,303

(22) PCT Filed: Jul. 31, 2008
(86) PCT No.: PCT/GB2008/002617

§ 371 Date: Jun. 30, 2010

Related U.S. Application Data
(60) Provisional application No. 60/935,270, filed on Aug. 3, 2007.

(51) Int. Cl. A61K 38/43; C07D 277/34; A61K 31/426; A61K 38/28; A61K 38/00; A61K 38/26; A61P 3/10; A61P 9/10; A61P 9/12; A61P 3/04; A61P 25/28; A61P 13/00

(52) U.S. Cl. 424/94.1; 548/184; 514/369; 514/5.9; 514/1.1; 514/7.2

(57) ABSTRACT

There is provided a compound of formula (I): wherein Y has the meaning given in the description. Such compounds are potentially useful in the treatment of disorders or conditions caused by, linked to, or contributed to by, excess adiposity, such as hyperinsulinemia and type 2 diabetes.

### FIELD OF THE INVENTION

This invention relates to pharmaceutically-useful compounds. The invention also relates to the use of such compounds in the treatment diabetes and associated conditions linked to excess adiposity and/or hyperinsulinemia and associated conditions.

### BACKGROUND

Elevated FFAs and hyperinsulinemia (hypersecretion of insulin) also represent new targets for treatment of obesity-related disorders/metabolic syndrome.

The metabolic syndrome has become increasingly common, and affects an estimated 47 million adults in the US alone. The syndrome is characterized by a combination of metabolic risk factors such as central obesity, atherogenic dyslipidemia, hypertension, insulin resistance or glucose intolerance. The syndrome is also characterised by hyperinsulinemia, a prothrombotic state in the blood, and a proinflammatory state.

Underlying causes of metabolic syndrome include obesity, physical inactivity and genetic factors. Sufferers are at an increased risk of coronary heart disease and other diseases related to the build up of plaques in artery walls, for example stroke, peripheral vascular disease and type 2 diabetes.

Diabetes is the most common metabolic disease with a high incidence in western countries, with more than 170 million people currently affected by type 2 diabetes. It is a chronic, presently incurable disease and sufferers have a high risk of developing life threatening complications as the disease progresses. The overall cost to society of diabetes and its complications is huge.

Over 300 million people worldwide suffer from obesity, with at least 1 billion people being regarded as overweight. Both problems are associated with elevated FFAs and hyperinsulinemia and can lead to increased insulin resistance and, in the worst case, the development of diabetes (approximately 80 percent of all adult diabetics are overweight), metabolic syndrome, fatty liver and/or other conditions or diseases.

Thus, to a large extent, obesity, metabolic syndrome and diabetes are interrelated and there is a substantial need for better pharmacological treatment of patients with one or more of these conditions.

Insulin is both a potent hormone and growth factor. In addition to obesity, hyperinsulinemia is apparent in conditions such as impaired glucose tolerance, early or mild type 2 diabetes, polycystic ovary syndrome and Alzheimer's disease. Evidence is accumulating that hyperinsulinemia plays a major role in the development of these diseases.

Elevated plasma FFAs stimulate pancreatic β-cells and is one cause of hyperinsulinemia. A medicament that modulates (e.g. suppresses) the stimulatory effect by FFA on insulin secretion may therefore represent a novel therapeutic strategy to treat or prevent disorders caused by, linked to, or contributed to by, hyperinsulinemia.

A possible mechanism that may underpin the cause of the development of hyperinsulinemia after exposure of elevated plasma FFAs may be explained by Steneberg et al (2005), Cell Metabolism, 1, 245-258, which reports a study under high fat dietary conditions, and suggests that GPR40 may play a pivotal role in the pathogenic process leading to diabetes. A mouse mutant lacking the GPR40 receptor was protected from the disease.

Another FFA receptor, GPR120, is expressed abundantly in a variety of tissues, especially the intestinal tract. The stimulation of GPR120 by FFAs promotes the secretion of GLP-1 and increases circulating insulin (see Hirasawa et al (2005), Nature Medicine, 11, 90-94).

No existing therapies for the different forms of diabetes appear to reduce hyperinsulinemia:
(a) insulin secretagogues, such as sulphonylureas stimulate only the insulin secretion step;
(b) metformin mainly acts on glucose production from the liver;
(c) peroxisome proliferator-activated receptor-γ (PPAR-γ) agonists, such as the thiazolidinediones, enhance insulin action; and
(d) α-glucosidase inhibitors interfere with gut glucose production.

All of these therapies fail to arrest progression of the disease and, over time, also fail to normalize glucose levels and/or to stop subsequent complications.

More recent therapies for the treatment of type 2 diabetes have limitations. For example, exenatide needs to be administered by subcutaneous injection and also has storage stability shortcomings.

Furthermore, existing therapies for the treatment of type 2 diabetes are known to give rise to undesirable side effects. For example, insulin secretagogues and insulin injections may cause hypoglycaemia and weight gain. Patients may also become unresponsive to insulin secretagogues over time. Metformin and α-glucosidase inhibitors often lead to gastrointestinal problems and PPAR-γ agonists tend to cause increased weight gain and oedema. Exenatide is also reported to cause nausea and vomiting.

With the epidemic increase in obesity in western society there is an urgent unmet clinical need to develop novel innovative strategies with the aim to suppress the detrimental effects of excess adiposity and hyperinsulinemia without causing hyperglycemia and diabetes. Further, there is a clear need for new drugs with a superior effect and/or less side effects.

Polycystic ovary syndrome (PCOS) is one of the most common endocrine disorders in the human, affecting approximately 10% of women of reproductive age. The syndrome is associated with a wide range of endocrine and metabolic abnormalities, including insulin resistance (see Ehrmann at al (2006), J Clin Endocrinol Metab, January 91 (1), 48-53). PCOS patients are typically hyperinsulinemic and insulin resistant. Hyperinsulinemia may contribute to hyperandrogenic, anovulatory dysfunction via a multitude of ways. In vitro and in vivo studies suggest that insulin synergizes with LH to promote androgen production by thecal cells. Insulin inhibits hepatic synthesis of sex hormone binding globulin, thereby increasing the free pool of androgens (Nestler (1997), Hum Reprod., October 12, Suppl 1, 53-62).

In Alzheimer's disease (AD), longitudinal studies have established a strong association with hyperinsulinemia. Hyperinsulinemia is also related to a significant decline in memory-related cognitive scores, but not to decline in other cognitive domains. Thus, hyperinsulinemia is associated with a higher risk of AD and decline in memory.

Insulin-degrading enzyme also appears to constitute a mechanistic link between hyperinsulinemia and AD (Wei and Folstein (2006), Neurobiology of Aging, 27, 190-198). This enzyme degrades both insulin and amyloid-β (Aβ) peptide, a short peptide found in excess in the AD brain. Evidence suggests that hyperinsulinemia may elevate Aβ through insulin's competition with the latter for insulin-degrading enzyme. Formation of neurofibrillary tangles, which contain hyperphosphorylated tau, represents a key step in the pathogenesis of neurodegenerative diseases. Promoting peripheral insulin stimulation, rapidly increased insulin receptor tyrosine phosphorylation, mitogen-activated protein kinase and phosphatidylinositol (PI) 3-kinase pathway activation, and dose-dependent tau phosphorylation at Ser(202) in the central nervous system in an insulin receptor-dependent manner.

Thus, peripherally injected insulin directly targets the brain and causes rapid cerebral insulin receptor signal transduction, revealing an additional link between hyperinsulinemia and neurodegeneration.

Studies on patients suffering from Systemic Lupus Erythematosus (SLE) have shown that these patients have significantly higher fasting insulin levels compared to healthy controls. They also have increased risk of coronary heart disease (CHD) which is not fully explained by the classic CHD risk factors, Magadmi et al (2006) J Rheumatol., January 33, 50-56. Thus, hyperinsulinemia may be a treatable risk factor in non-diabetic and diabetic SLE patients. Recent studies on metabolic syndrome in patients with chronic kidney disease suggest that insulin resistance and hyperinsulinemia are independently associated with an increased prevalence of the disease. Insulin per se can promote the proliferation of mesangial cells and the production of matrix proteins, and also stimulates the expression of growth factors such as IGF-1 and TGF-β, that are involved in mitogenic and fibrotic processes in nephropathy. Insulin also interferes with the systemic RAS and specifically increases the effect of angiotensin II on mesangial cells. Hyperinsulinemia also increases levels of endothelin-1 and is associated with increased oxidative stress. In conclusion, reduction of hyperinsulinemic levels may be of therapeutic value for patients with progressive renal disease (e.g. chronic renal failure; Sarafidis and Ruilope (2006), Am J Nephrol, 26, 232-244).

AMPK is a protein kinase enzyme that consists of three protein sub-units and plays a role in cellular energy homeostasis. The activation of AMPK triggers several biological effects, including the inhibition of cholesterol synthesis, lipogenesis, triglyceride synthesis, and the reduction of hyperinsulinemia. AMPK is also involved in a number of pathways that are important in cancer.

Current anti-diabetic drugs (e.g. metformin) are known to not be significantly potent AMPK activators, but only activate AMPK indirectly and with low efficacy. However, due to the biological effects of AMPK activation at the cell level, compounds that are AMPK activators, and preferably direct activators of AMPK, may find utility as anti-diabetic drugs.

Studies show that fibrosis is involved in many pathological states in the body (T. A. Wynn (2008) J. Pathology 214, 199-210. It has been shown that AMPK negatively regulates TGFβ-stimulated myofibroblast transdifferentiation and may therefore play a role in disorders where fibrosis develops (Misrha et al (2008), J. Bio. Chem. 283, 10461-10469). The resulting reduction of collagen may be of therapeutic value in any disease state or condition where fibrosis plays a role.

The listing or discussion of an apparently prior-published document in this specification should not necessarily be taken as an acknowledgement that the document is part of the state of the art or is common general knowledge.

U.S. Pat. No. 1,293,741 discloses inter alia thiazolidinones. However, there is no mention of the use of the compounds disclosed therein in the treatment of diabetes.

U.S. Pat. No. 4,103,018 and U.S. Pat. No. 4,665,083 disclose inter alia thiazolidinones. However, there is no mention or suggestion of thiazolidinones that are substituted in the 5-position.

WO 2005/051890 discloses inter alia thiazolidinones (which are ultimately substituted with a cyclopropyl group) that may be useful in the treatment of diabetes. However, there is no mention or suggestion in this document of thiazolidinones that are substituted in the 5-position with a benzyl group.

EP 1 535 915 discloses various furan and thiophene-based compounds.

EP 1 559 422 discloses a huge range of compounds for use in the treatment of inter alia cancer. However, this document does not appear to relate to thiazolidinones.

US patent application US 2006/0089351 discloses various benzothiazole derivatives as neuropeptide Y receptor antagonists, and therefore of use in the treatment of eating disorders. International patent application WO 2006/020680 discloses a vast range of heterocyclic compounds as modulators of nuclear receptors.

International patent applications WO 2005/075471 and WO 2005/116002 disclose inter alia thiazolidinones and oxazolidinones as 11-β-hydroxysteroid dehydrogenase type 1 inhibitors. There is no mention or suggestion of thiazolidinones or oxazolidinones that are each substituted at the 5-position with a benzyl group.

International patent application WO 2006/040050 discloses certain quinazolinylmethylene thiazolinones as CDK1 inhibitors. Similarly, US patent application US 2006/0004045 discloses quinolinylmethylene thiazolinones.

International patent applications WO 2007/010273 and WO 2007/010281 both disclose e.g. thiazolidin-4-one compounds that are able to antagonize the stimulatory effect of FFAs on cell proliferation when tested in an assay using a human breast cancer cell line (MDA-MB-231). Such compounds are thus indicated in the treatment of cancer and/or as modulators of FFAs.

According to the invention therefore, there is provided the compound 5-(3-(trifluoromethyl)benzyl)-2-(3,4-dichlorophenyl)sulfonyl-iminothiazolidin-4-one:

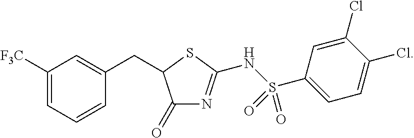

Another compound that may be mentioned includes 3,4-dichloro-N-[1,1-dioxo-5-(3-trifluoromethylbenzyl)-1,5-dihydro-1λ⁶-[1,4,2]dithiazol-3-yl]-benzenesulfon-amide:

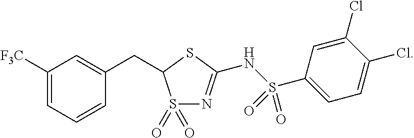

Hence, the compounds of the invention include the following compounds of formula I,

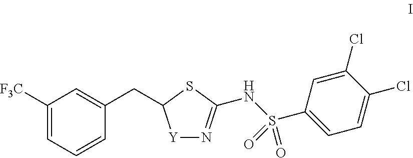

wherein:
Y represents —C(O)— or —S(O)₂—,
or a pharmaceutically-acceptable salt or solvate, or a pharmaceutically functional derivative thereof.

Pharmaceutically-acceptable salts that may be mentioned include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable addition salts include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, and arylsulphonic acids.

“Pharmaceutically functional derivatives” of compounds of formula I as defined herein includes ester derivatives and/or derivatives that have, or provide for, the same biological function and/or activity as any relevant compound. Thus, for the purposes of this invention, the term also includes prodrugs of compounds of formula I.

The term “prodrug” of a relevant compound of formula I includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of formula I may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula I wherein a hydroxyl, amino, sulfhydryl, carboxy or carbonyl group in a compound of formula I is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of formula I, as well as pharmaceutically-acceptable salts, solvates and pharmaceutically functional derivatives of such compounds are, for the sake of brevity, hereinafter referred to together as the “compounds of formula I”.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention. For example, the following tautomers are included within the scope of the invention:

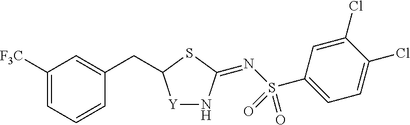

In such compounds, the relevant proton may be attached to either of the two different nitrogen atoms, and the ‘proton shift’ may be accompanied by one or more double bond shift.

Compounds of formula I may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Compounds of formula I may be prepared by:

(i) for compounds of formula I in which Y represents —C(O)—, reaction of either:
(A) a compound of formula II,

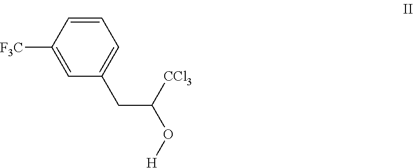

(B) a compound of formula III,

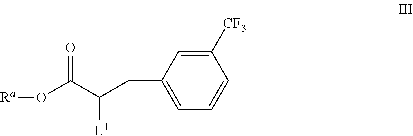

wherein R^{a} represents C₁₋₆ alkyl (e.g. methyl or, preferably, ethyl; so forming an ester group), L¹ represents a suitable leaving group, such as a sulfonate group (e.g. mesylate or, preferably, tosylate) or, for example preferably, halo (e.g. bromo or chloro); or
(C) a compound of formula IV,

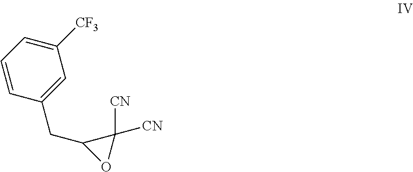

with, in each case, a compound of formula V,

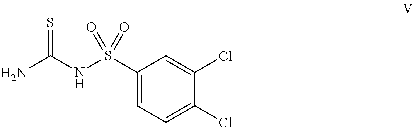

under reaction conditions known to those skilled in the art, for example for reaction (A) above conditions such as those described in Blanchet et al, Tetrahedron Letters, 2004, 45, 4449-4452; for reaction (B) above, conditions such as those described in St. Laurent et al, Tetrahedron Letters, 2004, 45, 1907-1910; K. Arakawa et al., Chem. Pharm. Bull. 1997, 45, 1984-1993; A. Mustafa, W. Musker, A. F. A. M. Shalaby, A. H. Harhash, R. Daguer, Tetrahedron 1964, 20; 25-31; or P. Herold, A. F. Indolese, M. Studer, H. P. Jalett, U. Siegrist, H. U. Blaser, Tetrahedron 2000, 56, 6497-6499 and for reaction (C) above, conditions such as those described in Le Martchalal et al, Tetrahedron 1990, 46, 453-464;
(ii) for compounds in which, preferably, Y represents —S(O)₂—, reaction of a compound of formula VI,

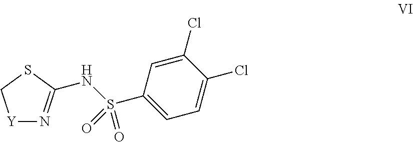

wherein Y is as hereinbefore defined and preferably represents —S(O)₂—, with a compound of formula VII,

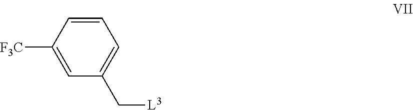

wherein L³ represents a suitable leaving group (e.g. a halo, such as chloro, iodo or, preferably, bromo, or a sulfonate group), under reaction conditions known to those skilled in the art, for example, in the presence of a suitable base (e.g. an organometallic base (e.g. an organolithium, for example a weak organolithium base such as lithium hexamethyldisilazide (LHMDS)), an alkali metal base (e.g. sodium hydride) or an amide salt (e.g. (Me₃Si)₂NNa) and the like) and a suitable solvent (e.g. tetrahydrofuran, dimethylformamide, dimethylsulfoxide or the like) at room temperature or below (such as at sub-zero temperatures (e.g. −78° C.)). For example, for the synthesis of such compounds in which Y represents —S(O)₂— and/or W represents a direct bond, reaction conditions include those described in Zbirovsky and Seifert, Coll. Czech. Chem. Commun. 1977, 42, 2672-2679 or Von Zaki El-Heweri, Franz Runge, Journal fü praktische Chemie, 4, Band 16, 1962;
(iii) reaction of a compound of formula VIII,

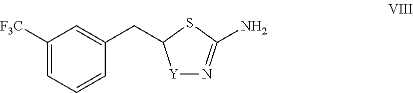

in which Y is as hereinbefore defined, with a compound of formula IX,

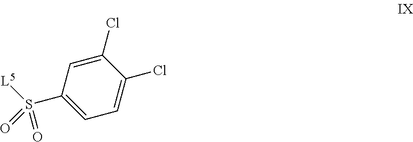

wherein L⁵ represents a suitable leaving group such as halo (e.g. chloro), under reaction conditions known to those skilled in the art, for example in the presence of a suitable base (e.g. NaH, NaOH, triethylamine, pyridine, sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and solvent (e.g. pyridine (which may serve as the base and solvent) DMF or dichloromethane (e.g. further in the presence of water and, optionally, a phase transfer catalyst)) for example at room temperature e.g. as described in Hurst, D. T.; Stacey, A. D., Nethercleft, M., Rahim, A., Harnden, M. R. Aust. J. Chem. 1998, 41, 1221;
(iv) for compounds of formula I in which Y represents —S(O)₂—, reaction of a compound of formula I×A,

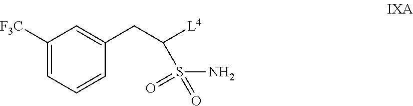

wherein L⁴ represents a suitable leaving group, such as halo (e.g. chloro), with a compound of formula XIII as described hereinafter, under reaction conditions known to those skilled in the art;
(v) for compounds of formula I in which Y preferably represents —S(O)₂—, reaction of a compound of formula IXB,

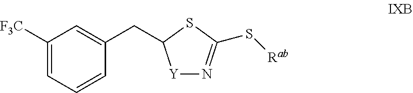

wherein R^{ab} represents C₁₋₆ alkyl (e.g. C₁₋₂ alkyl, such as methyl) optionally substituted by one or more halo atoms (but preferably unsubstituted) and Y preferably represents —S(O)₂—, with 3,4-dichlorobenzenesulfonamide, under reaction conditions known to those skilled in the art.

The compound of formula II may be prepared by reaction of a compound of formula X,

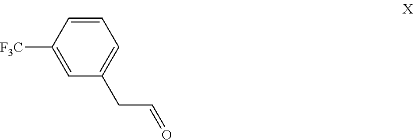

with trichloroacetic acid under standard conditions known to those skilled in the art, for example such as those described in the journal article mentioned in respect of preparation of compounds of formula I (process step (i) (part (A)) above).

Compounds of formula III may be commercially available, prepared under standard conditions or, for those compounds in which L¹ represents a halo group, by reaction with 3-trifluoromethylaniline to form the corresponding diazonium salt (for example by reaction with sodium nitrite at low temperatures such as at about 0° C.) followed by reaction with a compound of formula XI,

R^{a}—OC(O)CH═CH₂ XI

wherein R^{a} is as defined above, in the presence of a suitable solvent (e.g. acetone) and a hydrohaiic acid which is preferably concentrated (e.g. in the case where L¹ represents chloro, concentrated hydrochloric acid) optionally in the presence of an agent that aids the Michael addition of the halide onto the acrylate/enone such as cuprous oxide.

Compounds of formula III in which L¹ represents a sulfonate group (e.g. a toslyate or mesylate) may be prepared by reaction of a compound corresponding to a compound of formula III but in which L¹ represents —OH with an appropriate sulfonyl chloride (e.g. tosyl chloride or mesyl chloride) under standard conditions known to those skilled in the art, for example at around room temperature, in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, 1,8-diazabicyclo-[5.4.0]undec-7-ene, sodium hydroxide, or mixtures thereof), an appropriate solvent (e.g. pyridine, dichloromethane, chloroform, tetrahydrofuran, dimethylformamide, triethylamine, dimethylsulfoxide, water or mixtures thereof) and, in the case of biphasic reaction conditions, optionally in the presence of a phase transfer catalyst.

Compounds of formula VI in which Y represents —S(O)₂— may be prepared by reaction of a compound of formula XII,

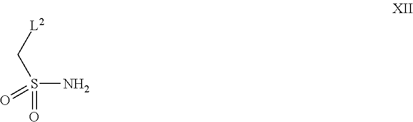

wherein L² represents a suitable leaving group, such as halo (e.g. chloro), either with:
(i) a compound of formula XIII,

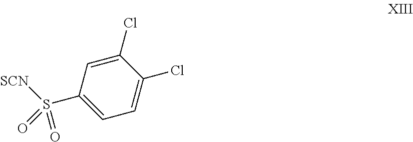

under conditions known to those skilled in the art, for example such as those described in Zbirovsky and Seifert, Coll. Czech. Chem. Commun. 1977, 42, 2672-2679 or Von Zaki El-Hewed, Franz Runge, Journal für praktische Chemie, 4, Band 16, 1962, e.g. in the presence of base (e.g. an aqueous solution of NaOH) in an appropriate solvent (e.g. acetone), for example at elevated temperature (e.g. 50°); or
(ii) a compound of formula V as hereinbefore described, under conditions known to those skilled in the art.

Compounds of formula VIII may be prepared by reaction of a compound of formula IXB as hereinbefore described with ammonia, under standard conditions known to those skilled in the art.

Compounds of formula IXB may be prepared by reaction of a compound of formula XIIIA,

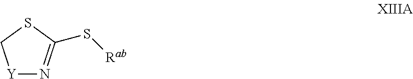

wherein Y is as hereinbefore defined, but preferably represents —S(O)₂— and R^{ab} is as hereinbefore defined, with a compound of formula VII as hereinbefore defined, for example under reaction conditions such as those hereinbefore described in respect of preparation of compounds of formula I (process step (ii) above).

Compounds of formula XII may be prepared by reaction of a compound of formula XIV,

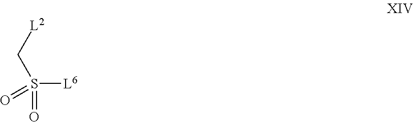

wherein L⁶ represents a suitable leaving group such as halo (e.g. chloro) and L² is as hereinbefore defined, with ammonia (e.g. in gaseous or other form) for example under standard conditions known to those skilled in the art, such as those described in respect of preparation of the precursors to compounds of formula I above (process step (vi) above) or, preferably, in the presence of diethyl ether at low temperature (e.g. about 0° C.) in which case the skilled person will appreciate that the ammonia additionally serves as a base.

Compounds of formulae IV, V, VII, VIII, IX, IXA, IXB, X, XI, XIII, XIIIA and XIV (and also certain compounds of e.g. formula II and III) are either commercially available, are known in the literature, or may be obtained either by analogy with the processes described herein (or processes described in references contained herein), or by conventional synthetic procedures, in accordance with standard techniques, from available starting materials using appropriate reagents and reaction conditions. For example, the compound of formula XIII may be prepared in accordance with the procedure described in H. Hartke Arch. Pharm. 1966, 299 (2), 174-178 (i.e. by the reaction of 3,4-dichlorobenzenesulfonamide with CS₂, in the presence of KOH (2 equivs.) and DMF, so forming dipotassium 3,4-dichloro-N-dimercaptomethylene-benzenesulfonamide, which may in turn be reacted with Cl₂C(O) in the presence of ether to form the compound of formula XIII). The compound of formula XIIIA may be prepared in accordance with the procedures described in U.S. Pat. No. 3,345,374 (e.g. Examples 13 and 27). Further, the compound of formula V may be prepared in accordance with the procedure described in Beuchet et al, Eur. J. Med. Chem. 34 (1999), 773-779 (i.e. by the reaction of 3,4-dichlorobenzene-sulfonyl chloride with NaNHCN in the presence of ether, followed by reaction with Na₂S₂O₃/H₂SO₄/H₂O).

Substituents such as Y in final compounds of formula I (or precursors thereto and other relevant intermediates) may be modified one or more times, after or during the processes described above by way of methods that are well known to those skilled in the art. Examples of such methods include oxidations, substitutions, reductions, alkylations, acylations, hydrolyses, esterifications, and etherifications. The precursor groups can be changed to a different such group, or to the groups defined in formula I, at any time during the reaction sequence.

Compounds of formula I may be isolated from their reaction mixtures using conventional techniques.

It will be appreciated by those skilled in the art that, in the processes described hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Chemistry”, edited by J W F McOmie, Plenum Press (1973), and “Protective Groups in Organic Synthesis”, 3^{rd} edition, T. W. Greene & P. G. M. Wutz, Wiley-Interscience (1999).

As used herein, the term “functional groups” means, in the case of unprotected functional groups, hydroxy-, thiolo-, aminofunction, carboxylic acid and, in the case of protected functional groups, lower alkoxy, N—, O—, S— acetyl, carboxylic acid ester.

### Medical and Pharmaceutical Uses

Compounds of formula I are indicated as pharmaceuticals. According to a further aspect of the invention there is provided a compound of formula I, or a pharmaceutically-acceptable salt or solvate, or a pharmaceutically functional derivative thereof, for use as a pharmaceutical.

Advantageously, compounds of formula I may be AMPK agonists, i.e. they may activate AMPK. By ‘activate AMPK’, we mean that the steady state level of phosphorylation of the Thr-172 moiety of the AMPK-α subunit is increased compared to the steady state level of phosphorylation in the absence of the agonist. Alternatively, or in addition, we mean that there is a higher steady state level of phosphorylation of any other proteins downstream of AMPK, such as acetyl-CoA carboxylase (ACC).

As the compounds of formula I may be AMPK activators, they may therefore be useful in the treatment of diseases such as those described herein, especially diabetes (e.g. type II diabetes).

Compounds of formula I are therefore indicated for use in the treatment of a disorder or a condition caused by, linked to, or contributed to by, excess adiposity and/or hyperinsulinemia.

According to a further aspect of the invention, there is provided the use of a compound of formula I, or a pharmaceutically-acceptable salt or solvate, or a pharmaceutically functional derivative thereof, for the manufacture of a medicament for the treatment of a disorder or condition caused by, linked to, or contributed to by, excess adiposity and/or hyperinsulinemia.

The term “disorder or condition caused by, linked to, or contributed to by, excess adiposity and/or hyperinsulinemia” will be understood by those skilled in the art to include hyperinsulinemia and associated conditions, such as type 2 diabetes, glucose intolerance, insulin resistance, metabolic syndrome, dyslipidemia, hyperinsulinism in childhood, hypercholesterolemia, high blood pressure, obesity, fatty liver conditions, diabetic nephropathy, diabetic neuropathy, diabetic retinopathy, cardiovascular disease, atherosclerosis, cerebrovascular conditions such as stroke, systemic lupus erythematosus, neurodegenerative diseases such as Alzheimer's disease, and polycystic ovary syndrome. Other disease states include progressive renal disease such as chronic renal failure.

Preferred disorders include hyperinsulinemia and, particularly, type 2 diabetes.

Certain compounds of formula I may also have the additional advantage that they exhibit partial agonist activity and may therefore be useful in conditions, such as late type 2 diabetes, in which stimulation of the production of insulin is required. By “agonist activity”, we include direct and indirect-acting agonists.

According to a further aspect of the invention there is provided a method of treatment of a disorder or a condition caused by, linked to, or contributed to by, excess adiposity and/or hyperinsulinemia, which method comprises the administration of an effective amount of a compound of formula I, or a pharmaceutically-acceptable salt or solvate, or a pharmaceutically functional derivative thereof, to a patient in need of such treatment.

Compounds of formula I may also be of use in the treatment of cancer (primary and metastatic cancers).

The term “cancer” will be understood by those skilled in the art to include one or more diseases in the class of disorders that is characterized by uncontrolled division of cells and the ability of these cells to invade other tissues, either by direct growth into adjacent tissue through invasion, proliferation or by implantation into distant sites by metastasis.

Compounds of formula I may reduce the rate of cell proliferation when tested in an assay using a human breast cancer cell line (e.g. MDA-MB-231). The compounds may thus possess a beneficial inhibitory effect on the ability of tumors of this type, and of cancers generally, to survive. Compounds of formula I may also reduce the rate of cell proliferation when tested in other cancer cells lines such as MCF-7, PC-3, Jurkat, Skov-3, HL60, MV4-11, HT29, K562, MDA-MB231, HCT116 wt, HCT116P53−/−, A-549, DU-145, LOVO, HCT-116 and PANC-1.

In a preferred embodiment, compounds of formula I are capable of inhibiting the proliferation of cancer cells. By “proliferation” we include an increase in the number and/or size of cancer cells.

Alternatively, or preferably in addition, compounds of formula I are capable of inhibiting metastasis of cancer cells.

By “metastasis” we mean the movement or migration (e.g. invasiveness) of cancer cells from a primary tumor site in the body of a subject to one or more other areas within the subject's body (where the cells can then form secondary tumors). Thus, in one embodiment the invention provides compounds and methods for inhibiting, in whole or in part, the formation of secondary tumors in a subject with cancer. It will be appreciated by skilled persons that the effect of a compound of formula I on “metastasis” is distinct from any effect such a compound may or may not have on cancer cell proliferation.

Advantageously, compounds of formula I may be capable of inhibiting the proliferation and/or metastasis of cancer cells selectively.

By “selectively” we mean that the combination product inhibits the proliferation and/or metastasis of cancer cells to a greater extent than it modulates the function (e.g. proliferation) of non-cancer cells. Preferably, the compound inhibits the proliferation and/or metastasis of cancer cells only.

Compounds of formula I may be suitable for use in the treatment of any cancer type, including all tumors (non-solid and, preferably, solid tumors). For example, the cancer cells may be selected from the group consisting of cancer cells of the breast, bile duct, brain, colon, stomach, reproductive organs, thyroid, hematopoietic system, lung and airways, skin, gallbladder, liver, nasopharynx, nerve cells, kidney, prostate, lymph glands and gastrointestinal tract. Preferably, the cancer is selected from the group of colon cancer (including colorectal adenomas), breast cancer (e.g. postmenopausal breast cancer), endometrial cancer, cancers of the hematopoietic system (e.g. leukemia, lymphoma, etc), thyroid cancer, kidney cancer, oesophageal adenocarcinoma, ovarian cancer, prostate cancer, pancreatic cancer, gallbladder cancer, liver cancer and cervical cancer. More preferably, the cancer is selected from the group of colon, prostate and, particularly, breast cancer. Where the cancer is a non-solid tumor, it is preferably a hematopoietic tumor such as a leukemia (e.g. Acute Myelogenous Leukemia (AML), Chronic Myelogenous Leukemia (CML), Acute Lymphocytic Leukemia (ALL), Chronic Lymphocytic Leukemia (CLL).

Preferably, the cancer cells are breast cancer cells.

Compound of formula I may also be of use in the treatment of a condition/disorder where fibrosis plays a role. Compounds of formula I may also be useful in the treatment of sexual dysfunction (e.g. the treatment of erectile dysfunction).

A condition/disorder where fibrosis plays a role includes (but is not limited to) scar healing, keloids, scleroderma, idiopathic pulmonary fibrosis, systemic sclerosis, liver cirrhosis, eye macular degeneration, retinal and vitreal retinopathy, Crohn's/inflammatory bowel disease, post surgical scar tissue formation, radiation and chemotherapeutic-drug induced fibrosis, and cardiovascular fibrosis.

For the avoidance of doubt, in the context of the present invention, the terms “treatment”, “therapy” and “therapy method” include the therapeutic, or palliative, treatment of patients in need of, as well as the prophylactic treatment and/or diagnosis of patients which are susceptible to, the relevant disease states.

“Patients” include mammalian (including human) patients.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

In accordance with the invention, compounds of formula I may be administered alone, but are preferably administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Preferred modes of delivery include oral, intravenous, cutaneous or subcutaneous, nasal, intramuscular, or intraperitoneal delivery.

Compounds of formula I will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science 249, 1527 (1990).

Otherwise, the preparation of suitable formulations may be achieved non-inventively by the skilled person using routine techniques and/or in accordance with standard and/or accepted pharmaceutical practice.

Another aspect of the present invention includes a pharmaceutical composition comprising a therapeutically effective amount of a compound of formula I, or a pharmaceutically-acceptable salt or solvate, or a pharmaceutically functional derivative thereof, in combination with a pharmaceutically acceptable excipient, such as an adjuvant, diluent or carrier.

The amount of compound of formula I in the formulation will depend on the severity of the condition, and on the patient, to be treated, as well as the compound(s) which is/are employed, but may be determined non-inventively by the skilled person.

Depending on the disorder, and the patient, to be treated, as well as the route of administration, compounds of formula I may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula I (or, if employed, a corresponding amount of a pharmaceutically acceptable salt or prodrug thereof).

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The compounds of formula I may also be used or administered in combination with one or more additional drugs useful in the treatment of disorders or conditions caused by, linked to, or contributed to by, excess adiposity and/or hyperinsulinemia, in combination therapy.

According to a further aspect of the invention, there is provided a combination product comprising:
(A) a compound of formula I; and
(B) another therapeutic agent useful in the treatment of a disorder or a condition caused by, linked to, or contributed to by, excess adiposity and/or hyperinsulinemia,
   wherein each of components (A) and (B) is formulated in admixture with a pharmaceutically-acceptable adjuvant, diluent or carrier.

Other therapeutic agents useful in the treatment of disorders or conditions caused by, linked to, or contributed to by, excess adiposity (such as hyperinsulinemia and type 2 diabetes) will be well known to those skilled in the art and include insulin, insulin secretagogues (such as sulphonylureas), metformin, peroxisome proliferator-activated receptor (PPAR) agonists (such as thiazolidinediones), α-glucosidase inhibitors, GLP-1 receptor agonists, DPP-IV inhibitors, exenatide, and inhibitors of 11-β hydroxysteroid dehydrogenase type 1. By “agonist” we include direct and indirect-acting agonists.

In one embodiment, the other therapeutic agent useful in the treatment of useful in the treatment of a disorder or a condition caused by, linked to, or contributed to by, excess adiposity (such as hyperinsulinemia and type 2 diabetes) may comprise GLP-1 or a biologically active fragment, variant, fusion of derivative thereof. For example, the agent may be selected from the group consisting of Exendin-4 (exenatide; Byetta), exenatide long acting release (LAR), exenatide derivatives (such as ZP10 developed by Zealand Pharmaceuticals), native GLP-1, human GLP-1 derivatives (such as BIM51077 (Ipsen and Roche)), DPP-IV resistant GLP-1 analogues (for example LY315902 and LY30761 SR (Lilly)), long acting GLP-1 derivatives (such as NN2211 (Liraglutide; Novo Nordisk)) and complex proteins (such as the GLP-1-albumin complex CJC-1131).

In an alternative embodiment, the other therapeutic agent useful in the treatment of a disorder or a condition caused by, linked to, or contributed to by, excess adiposity (such as hyperinsulinemia and type 2 diabetes) may comprise a dipeptidyl peptidase IV (DPP-IV) inhibitor. For example, the agent may be selected from the group consisting of Vildagliptin (LAF237), MK-0431-Sitagliptin and Saxagliptin.

In a further alternative embodiment, the other therapeutic agent useful in the treatment of useful in the treatment of a disorder or a condition caused by, linked to, or contributed to by, excess adiposity (such as hyperinsulinemia and type 2 diabetes) may comprise gastric inhibitory polypeptide (GIP), or a biologically active fragment, variant, fusion of derivative thereof. GIP, also known as glucose-dependent insulinotropic polypeptide, is a 42-amino acid peptide hormone synthesised in and secreted from K cells in the intestinal epithelium. An important determinant of GIP action is the N-terminal cleavage of the peptide to the inactive GIP (3-42). The enzyme DPP-4, which also cleaves GLP-1 and GLP-2, rapidly inactivates GIP both in vitro and in vivo. Hence, it may be desirable to administer GIP in combination with a DPP-4 inhibitor.

In a further alternative embodiment, the other therapeutic agent useful in the treatment of useful in the treatment of a disorder or a conditions caused by, linked to, or contributed to by, excess adiposity (such as hyperinsulinemia and type 2 diabetes) may comprise a selective inhibitor of 11-β hydroxysteroid dehydrogenase type 1 (11β-HSD1), an enzyme associated with conversion of cortisone to cortisol in the liver and adipose tissue. Examples of suitable 1113-HSD1 inhibitors/antagonists include AMG221 (developed by Amgen) and BVT83370 (developed by Biovitrum).

Combination products as described herein provide for the administration of compound of formula I in conjunction with the other therapeutic agent, and may thus be presented either as separate formulations, wherein at least one of those formulations comprises compound of formula I, and at least one comprises the other therapeutic agent, or may be presented (i.e. formulated) as a combined preparation (i.e. presented as a single formulation including compound of formula I and the other therapeutic agent).

Thus, there is further provided:

(1) pharmaceutical formulations including a compound of formula I; another therapeutic agent useful in the treatment of a disorder or a condition caused by, linked to, or contributed to by, excess adiposity and/or hyperinsulinemia; and a pharmaceutically-acceptable adjuvant, diluent or carrier; and
(2) kits of parts comprising components:
(a) a pharmaceutical formulation including a compound of formula I in admixture with a pharmaceutically-acceptable adjuvant, diluent or carrier; and
(b) a pharmaceutical formulation including another therapeutic agent useful in the treatment of a disorder or a condition caused by, linked to, or contributed to by, excess adiposity and/or hyperinsulinemia, in admixture with a pharmaceutically-acceptable adjuvant, diluent or carrier,
   which components (a) and (b) are each provided in a form that is suitable for administration in conjunction with the other.

Components (a) and (b) of the kits of parts described herein may be administered simultaneously or sequentially.

According to a further aspect of the invention, there is provided a method of making a kit of parts as defined above, which method comprises bringing component (a), as defined above, into association with a component (b), as defined above, thus rendering the two components suitable for administration in conjunction with each other.

By bringing the two components “into association with” each other, we include that components (a) and (b) of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:

(I) one of components (a) and (b) as defined herein; together with
(II) instructions to use that component in conjunction with the other of the two components.

The kits of parts described herein may comprise more than one formulation including an appropriate quantity/dose of compound of formula I, and/or more than one formulation including an appropriate quantity/dose of the other therapeutic agent, in order to provide for repeat dosing. If more than one formulation (comprising either active compound) is present, such formulations may be the same, or may be different in terms of the dose of either compound, chemical composition(s) and/or physical form(s).

With respect to the kits of parts as described herein, by “administration in conjunction with”, we include that respective formulations comprising compound of formula I and the other therapeutic agent are administered, sequentially, separately and/or simultaneously, over the course of treatment of the relevant condition.

Thus, in respect of the combination product according to the invention, the term “administration in conjunction with” includes that the two components of the combination product (compound of formula I and the other therapeutic agent) are administered (optionally repeatedly), either together, or sufficiently closely in time, to enable a beneficial effect for the patient, that is greater, over the course of the treatment of the relevant condition, than if either a formulation comprising compound of formula I, or a formulation comprising the other therapeutic agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two formulations may be administered (optionally repeatedly) prior to, after, and/or at the same time as, administration with the other component. When used in this context, the terms “administered simultaneously” and “administered at the same time as” include that individual doses of compound of formula I and the other therapeutic agent are administered within 48 hours (e.g. 24 hours) of each other.

The compounds/combinations/methods/uses described herein may have the advantage that, in the treatment of the conditions described herein, they may be more convenient for the physician and/or patient than, be more efficacious than, be less toxic than, have better selectivity, have a broader range of activity than, be more potent than, produce fewer side effects than, or may have other useful pharmacological properties over, similar compounds, combinations, methods (treatments) or uses known in the prior art for use in the treatment of those conditions or otherwise, for example over the compounds disclosed in international patent applications WO 2007/010273 and WO 2007/010281.

Further, such advantages may stem from the compounds of formula I being AMPK activators (e.g. especially where it is stated that the compounds described herein may have better selectivity, and may produce fewer side effects, e.g. gastrointestinal side effects).

Preferred, non-limiting examples which embody certain aspects of the invention will now be described, with reference to the following figures:

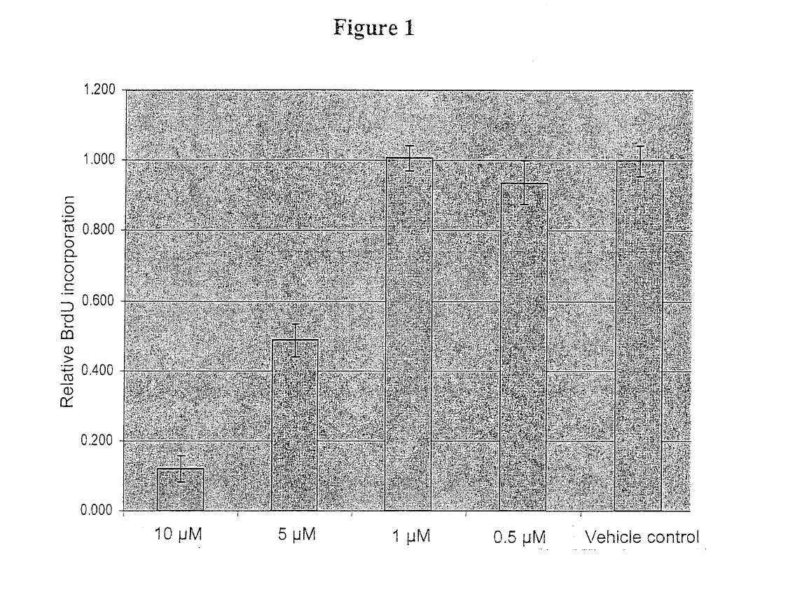

FIG. 1: Treatment of tumor cell lines with Example 1 generate a dose dependent reduction in proliferation in MDA-MB-231 human breast cancer cell lines as measured by BrdU incorporation.

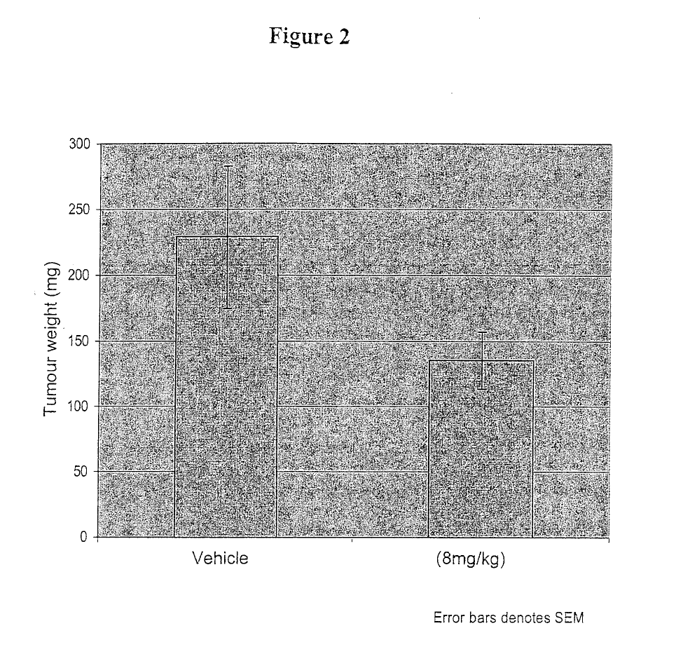

FIG. 2: Tumor weights of the group of mice treated with the vehicle control and of the group of mice treated with the compound of Example 1.

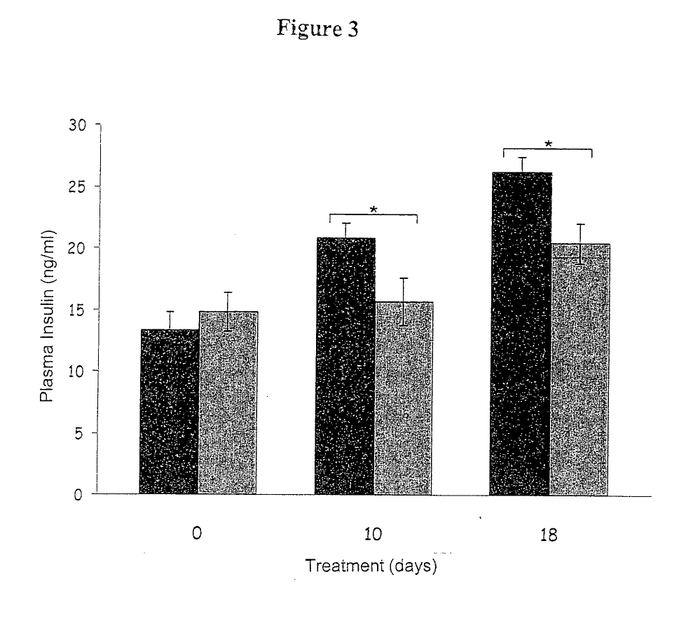

FIG. 3: Effect of compound of Example 1 on plasma insulin in vivo.

Plasma concentrations were compared after day 0, 16 h post dose of day 10 and 16 h post dose of day 18 of dosing with compound of Example 1 (30 mg/kg bodyweight, 5 ml/kg bodyweight, grey bar; i.e. the second relatively lighter coloured bar on the right hand side) or vehicle (5 ml/kg bodyweight, black bar; i.e. the first relatively darker coloured bar on the left hand side) in fed Ob/Ob mice (n=10 each). *Significantly different from vehicle control mice (p<0.05).

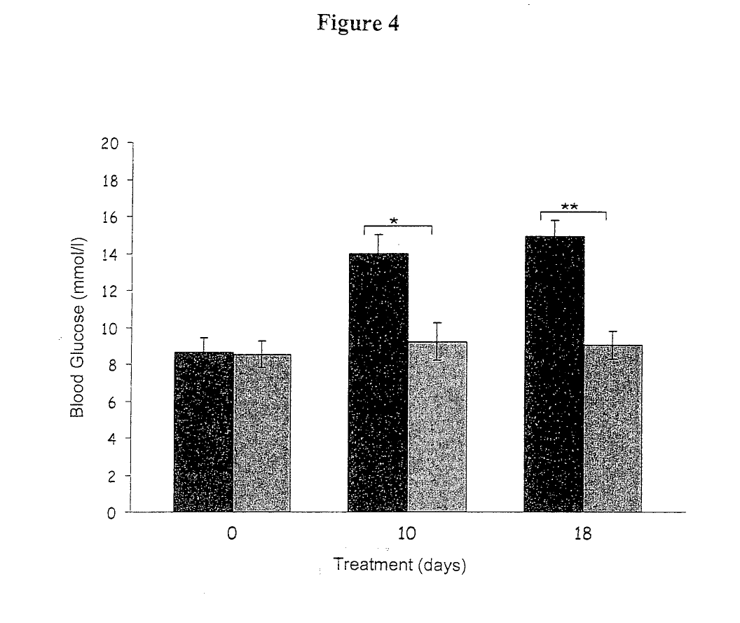

FIG. 4: Effect on compound of Example 1 on fed blood glucose in Ob/Ob mice. Fed blood glucose concentrations were measured at day 0, 16 h post dose of day 10 and 16 h post dose of day 18 of dosing in compound of Example 1 (30 mg/kg bodyweight, 5 ml/kg bodyweight, grey bar, i.e. the second relatively lighter coloured bar on the right hand side) or vehicle control (5 ml/kg bodyweight, black bar, i.e. the first relatively darker coloured bar on the left hand side) gavaged Ob/Ob mice (n=10 each). Significantly different from vehicle control mice **p<0.01; *** p<0.001.

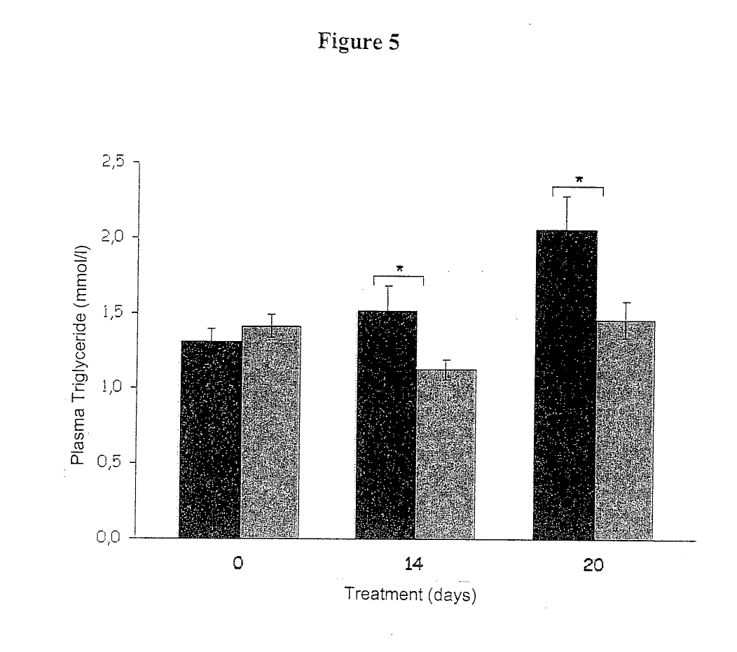

FIG. 5: Effect of compound of Example 1 on plasma triglycerides in vivo.

Plasma concentrations were compared after day 0, 16 h post dose of day 14 and 16 h post dose of day 20 of dosing with compound of Example 1 (15 mg/kg bodyweight, 2.5 ml/kg bodyweight, grey bar, i.e. the second relatively lighter coloured bar on the right hand side) or vehicle (2.5 ml/kg bodyweight, black bar, i.e. the first relatively darker coloured bar on the left hand side) in fed Ob/Ob mice (n=10 each). *Significantly different from vehicle control mice (p<0.05).

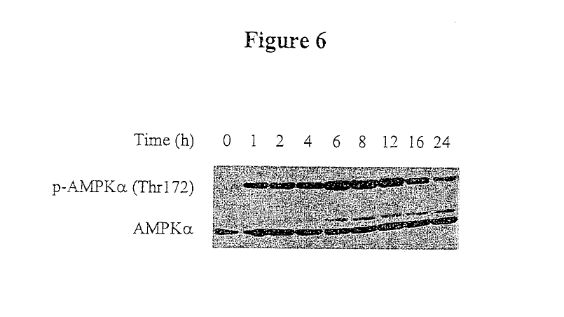

FIG. 6: Time course effect of the compound of Example 1 on AMPK phosphorylation.

HepG2 cells were quiesced in serum free DMEM overnight and treated with the compound of Example 1 for an additional 6 h. The phosphorylation of AMPK is shown in HepG2 cells exposed to compound of Example 1 (10 μM) for 1, 2, 4, 6, 8, 12, 16 and 24 h.

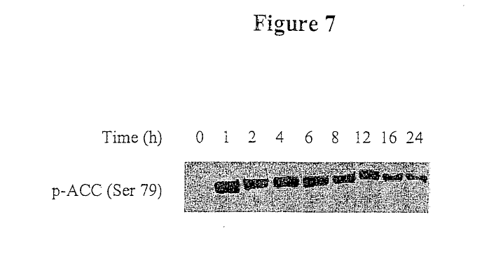

FIG. 7: Time course effect of the compound of Example 1 on ACC phosphorylation.

HepG2 cells were quiesced in serum free DMEM overnight and treated with compound of Example 1 for an additional 6 h. The phosphorylation of ACC is shown in HepG2 cells exposed to compound of Example 1 (10 μM) for 1, 2, 4, 6, 8, 12, 16 and 24 h.

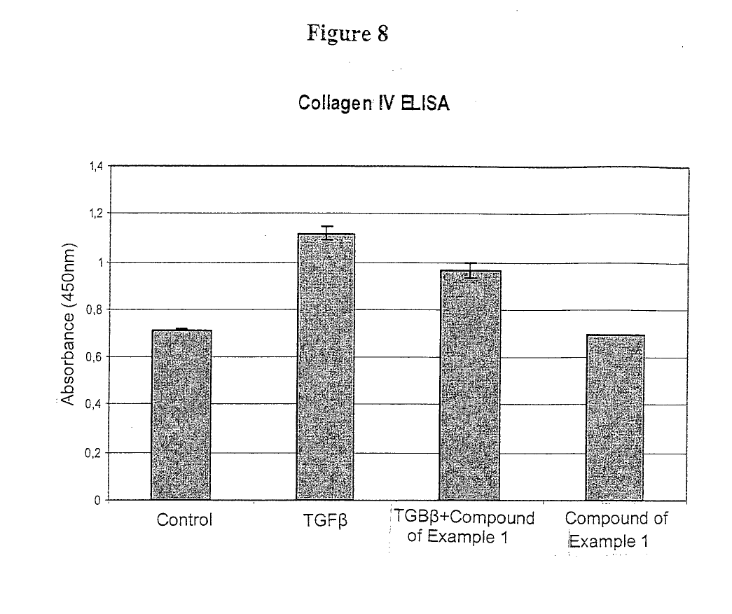

FIG. 8: The compound of Example 1 inhibits TGF-β-induced secretion of collagen IV in human primary mesangial cells, as indicated by the measurement of absorbance using the ELISA procedure

### EXAMPLES

The invention is illustrated by the following examples, in which the following abbreviations may be employed:

BrdU 5-bromo-2-deoxyuridine
DMF dimethylformamide
DMSO dimethylsulfoxide
ES electro spray
EtOAc ethyl acetate
LC liquid chromatography
MS mass spectrometry
NMR nuclear magnetic resonance
THF tetrahydrofuran

Where no preparative routes are includes, the relevant intermediate is commercially available (e.g. from Chemical Diversity, San Diego, Calif., USA or other available commercial sources).

### General Procedures

LC-MS was performed on a Sciex API 150 LC/ES-MS equipped with an ACE 3 C8 column (30×3.0 mm) using a flow of 1 mL/min. Two gradient systems of acetonitrile in water (with 0.1% TFA) were used for elution: A) 5-100% under 10 min, then 2 min 100% isocratic or B) 90-100% under 2 min, then 2 min 100% isocratic. Direct inlet ES-MS was also performed on a Bruker Esquire LC/ES-MS. ¹H nuclear magnetic resonance was recorded on a Bruker Avance DRX 400 spectrometer at 400.01 MHz using residual solvent as internal standard.

### Example 1

### 5-(3-(Trifluoromethyl)benzyl)-2-(3,4-dichlorophenyl)sulfonyliminothiazolidin-4-one

### (a) Methyl 2-chloro-3-(3-(trifluoromethyl)phenyl)propanoate

A solution of sodium nitrite (0.47 g, 6.82 mmol) in water (1.4 mL) was added drop-wise to a solution of 3-trifluoromethylaniline (0.77 mL, 6.21 mmol) in concentrated hydrochloric acid and acetone (14 mL), which mixture was prior cooled under an ice-water bath. The mixture was stirred at 0° C. for 10 min. After addition of methyl acrylate (3.37 mL, 37.4 mmol), cuprous oxide (40 mg) was added portion-wise to the mixture at 40° C. The mixture was stirred at 35° C. for 20 min and then washed twice with equal amounts of water and ethyl acetate (50 mL). The organic layer was dried with MgSO₄, filtered and concentrated. The crude oil was purified by silica gel chromatography using chloroform as eluent to give the sub-title compound (1.22 g, 4.58 mmol, 74%) as yellow oil. ES-MS m/z 289.1 (MNa+). NMR: δ(CDCl₃): 3.24 (dd, 1H), 3.43 (dd, 1H), 3.76 (s, 3H), 4.46 (dd, 1H), 7.4-7.6 (m, 4H).

### (b) 5-(3-(Trifluoromethyl)benzyl)-2-aminothiazol-4(5H)-one

Methyl 2-chloro-3-(3-(trifluoromethyl)phenyl)propanoate (2.44 g, 9.16 mmol; see step (a) above), thiourea (697 mg, 9.16 mmol), and NaOAc (848 mg, 10.13 mmol) were dissolved in 95% EtOH (20.0 mL). The mixture was heated at reflux for 20 h, after which the EtOH was evaporated. The crude product was washed with H₂O/CH₂Cl₂, and the solid separated was collected to give sub-title compound (1.5 g). The organic phase was dried, concentrated and triturated with isohexane to give more of the sub-title compound (0.5 g). The total mass of the crude sub-title compound was 2 g (7.29 mol, 80%). ES-MS m/z: 275 (MH+). The product was used for the next step without further purification.

### (c) [5-(3-(Trifluoromethyl)benzyl)-2-(3,4-dichlorophenyl)sulfonyliminothiazolid-in-4-one

To 5-(3-(trifluoromethyl)benzyl)-2-aminothiazol-4(5H)-one (1100 mg, 4.01 mmol) in dichloromethane (10 mL), was successively added pyridine (0.317 mL, 4.01 mmol) and 3,4-dichlorobenzenesulfonyl chloride (0.984 mg, 0.626 mL, 4.01 mmol). The reaction mixture was stirred at room temperature overnight and poured into a saturated aqueous solution of NaHCO₃. The water phase was extracted with CH₂Cl₂, and the organic phase was dried with MgSO₄, filtered and concentrated in vacuo. The crude material was purified by column chromatography using a gradient of CH₂Cl₂/MeOH (0-1%) as eluent to give 450 mg (0.931 mmol, 23.3%) of the title compound as colourless oil. Recrystallization from CH₂Cl₂/iso-hexane yielded 360 mg of a white-yellow solid. ES-MS: 506 (M+HNa) 481.0 (M−H). ¹H NMR: δ(400 MHz) (Acetone d₆): 3.25 (dd, 1H), 3.62 (dd, 1H), 4.82 (dd, 1H), 7.60-7.70 (m, 4H), 7.72-7.88 (m, 2H), 7.92 (d, 1H).

### Biological Tests

Descriptions of the cancer cell lines including source, tumor type, and morphology may be obtained from the American Type Culture Collection (ATCC) or its website (www.atcc.orq).

### Test A

### Cell Proliferation Assay

### Reagents

Dulbecco's modified Eagle's medium (D-MEM)+1000 mg/L Glucose

### +GlutaMAX™ 1+Pyruvate (Gibco #21885-025)

### V/V Foetal Bovine Serum (Gibco 10500-064)

5-bromo-2-deoxyuridine (BrdU)
Dimethyl sulfoxide (DMSO)

The cell line was propagated in D-MEM (Gibco 21885) supplemented with 10% Foetal calf serum. 15000 cells per well were seeded in 96 well plates and incubated overnight. The culture media was changed to serum-free D-MEM for 24 h. The culture media was then changed to serum free D-MEM containing either 0.2% DMSO as vehicle control or 10, 5, 1, 0.1 μM of the compound of Example 1 in 0.2% DMSO in quadruplicate. After 18 h incubation, BrdU was added according to manufacturer's recommendations. After 6 h incubation in the presence of BrdU, the culture media was removed and BrdU incorporation was measured using “Cell Proliferation ELISA, BrdU colorimetric” Roche (11647229001) according to manufacturer's recommendations.

### Results

Proliferation rate of MDA-MB-231 cells are reduced by relevant concentrations of the test compounds as measured by BrdU incorporation (see FIG. 1).

For example, in the above assay, the compound of Example 1, relative to the vehicle control (which displayed a BrdU incorporation of 1 unit) displayed the following (approximate) units of BrdU incorporations at different concentrations:

### 10 μM: 0.15

### 5 μM: 0.5

### 1 μM: 1

### 0.5 μM 0.95

These results are depicted in FIG. 1.

### Other Cell Proliferation Assays

The cell lines mentioned herein are also employed. For example:

### (i) PC-3;

### (ii) Jurkat; and

(iii) PANC-1.

### Results

Proliferation rate of cells in the cell lines (e.g. (i), (ii) or (iii)) is reduced by relevant concentrations of the test compounds as measured by BrdU incorporation (relative to a vehicle control). The reduction in proliferation may be dose dependent.

### Test B

### In Vivo Mouse Model—Test 1

5 week old Athymic BALB/cA nude mice are delivered from Taconic (Denmark) and kept under barrier conditions for 1 week acclimatisation. At 6 weeks, 17 mice are injected subcutaneously on the flank with 1.8×10⁶ MDA-MB-231 human breast cancer cells (LGC Promochem-ATCC) in a 50/50 v/v solution of phosphate buffered saline (PBS) (Gibco 10010-015, Invitrogen) Matrigel HC (BD Biosciences).

After 11 days, palpable tumors are observed in 16 mice. 2 mice are sacrificed and the tumors dissected and examined. 2 groups of 7 mice each are treated once daily by intraperitoneal injections of 1-10 mg/kg bodyweight of test compound in 79% PBS/20% Solutol HS 15 (BASF)/1% DMSO or vehicle control respectively for 5-30 days. The mice are sacrificed by cervical dislocation and tumors are dissected.

### Histology

The tumor tissue are fixated overnight in PBS (containing 4% w/v paraformaldehyde (Scharlau PA0095, Sharlau Chemie SA, Spain) at +4° C. The tumor tissue is then cryopreserved by 24 hour incubation in PBS containing 30% w/v sucrose (BDH #102745C (www.vwr.com) at +4° C. and embedded in Tissue-Tek embedding media (Sakura Finetek Europa BV, Netherlands). 10 μm cryosections are generated and stained with Mayers Hematoxylin (Dako) for 5 minutes and destained for 3×10 minutes in tap water. Slides are mounted using Dako faramount aqueous mounting medium and examined using a Nikon Eclipse TS 100 microscope documented using a Nikon coolpix 4500.

### Results

The tumors from mice treated with test compound and vehicle are analyzed for morphology by microscopic examination of hematoxylin stained cryosections.

Hematoxylin stained sections from tumors dissected from mice show that the cell-density in the interior of the tumors is reduced in tumors dissected from test compound treated mice as compared to tumors from vehicle treated mice, showing a correlation between treatment with test compound and reduction of cancer cells in xenograft tumors.

### In Vivo Mouse Model—Test 2

The above test procedure was followed, but 16 (rather than 17) mice were injected subcutaneously.

### Results

After 6 days, palpable tumors were observed in the 16 mice.

2 groups of 8 mice each were treated once daily by intraperitoneal injections of 8 mg/kg bodyweight of compound of Example 1 in 79% PBS/20% Solutol HS 15(BASF)/1% DMSO or vehicle control respectively for 27 days. Tumors were dissected and weighed at the end of the experiment.

The results are depicted in FIG. 2, where it can be seen that after the experiment the tumors of the group of mice treated with the vehicle control weighed approximately 225 mg, whereas the tumors of the group of mice treated with the compound of Example 1 weighed about 130 mg.

### Test C

### Insulin Measurement Study in Diabetic Ob/Ob Mice

### Method (I)

### Reagents

Ultra sensitive rat insulin ELISA kit (Crystal Chen inc) according to manufacturer's recommendations.

Serum insulin measurements on 8-9 week old Ob/Ob mice (Taconic) fasted for 4 h/unfasted/day after 4 h fast is performed. Mice are distributed to a vehicle control group (VC) or a test compound treatment group, so that mean s-insulin is equal between the groups. 1-20 mg/kg bodyweight of test compound in vehicle and VC groups are injected intraperitoneally or subjected to oral gavage once/twice daily for 2-4 weeks, after which serum insulin levels are measured as described above.

Alternatively, plasma insulin measurements on fed Ob/Ob mice (Taconic), is performed. Mice, 6-7 weeks of age are distributed to a vehicle control group (VC) or a test compound treatment group, so that the mean concentration of plasma insulin is equal between the groups. 1-30 mg/kg (e.g. 6 mg/kg) bodyweight of test compound in vehicle and VC groups are subjected to oral gavage twice daily for 18 days, after which plasma insulin levels are measured as described above.

### Results

Test compound attenuates hyperinsulinemia in Ob/Ob mice. The hyperinsulinemia observed in Ob/Ob mice is generally believed to be a consequence of obesity and perturbed lipid metabolism leading to insulin resistance. We interpret the activity of the test compound in Ob/Ob mice as attenuating the insulin resistance.

### Method (II)

### Aim

The aim of this study was to verify the efficacy of compound of Example 1 in the diabetic ob/ob mouse with regard to correction of the metabolic disorder hyperinsulinemia. Ob/ob mice were gavaged twice daily with compound of Example 1 and the effect of the compound on levels of plasma insulin were assessed and the results were compared to a concurrent control group gavaged with vehicle.

### Materials and Methods

### Materials

The compound of Example 1 was obtained from Isosep AB, Uppsala, Sweden. A stock solution of 6 mg/ml was prepared by dissolving the compound in PBS, pH 7.4, 1% DMSO and 0.5% methyl cellulose.

### Animals

Male B6.V-Lep^{ob}/JBomTac (model number OB-M) mice were bred and delivered by Taconic. Animals were housed in Umea University animal facility in transparent polycarbonate cages, with wood chip bedding at a 12 h light/darkness cycle, a temperature of −21° C., and a relative humidity of −50% throughout the accommodation and dosing periods. 5 animals were housed in each cage with free access to standard rodent chow (CRM(E)Rodent, Special Diets Services, Scanbur BK, Sweden) and tap water. All animal experiments were approved by the Local Ethics Review Committee on Animal Experiments, Umea Region.

### Animal Experimental Procedures

In vivo potency and efficacy were determined in groups of 10 mice. Male ob/ob mice, 6 to 7 weeks of age were gavaged (5 ml/kg bodyweight) twice-daily (8:00-9:00 A.M. and 4:00-5:00 P.M.) with compound of Example 1 (30 mg/kg bodyweight) for 18 days. Blood samples were drawn from the tail vein from fed animals at day 0, 10 and at day 18 of dosing for analysis of plasma insulin. Blood samples were collected into vials containing potassium-EDTA (Microvette CB300, Sarstedt). Plasma was separated by centrifugation at 4° C. and stored at −20° C. until assayed.

### Analytic Methods

Plasma insulin levels were determined according to the manufacturer's recommendations with a rat insulin ELISA kit using mouse insulin standard (Crystal Chem Inc).

### Data Analysis

Data in the figures are presented as means±SEM. P values were calculated using the Student's t-test. Values of P<0.05 (*) were considered to be statistically significant (P<0.01 ** and P<0.001 ***). Statistical analyses were performed using Microsoft Office Excel 2003.

### Results

Compound of Example 1 attenuates hyperinsulinemia in Ob/Ob mice, as shown by FIG. 3.

### Test D

### Blood Glucose Measurement Study in Diabetic Ob/Ob Mice

### Method (I)

### Reagents

Ascensia Elit XL (Bayer diagnostic) hand held glucometer.

Blood glucose measurements on 8-9 week old Ob/Ob mice (Taconic) fasted for 4 h/unfasted/day after 4 h fast is performed. Mice are distributed to a vehicle control group (VC) or a test compound treatment group, so that mean blood glucose is equal between the groups. 1-20 mg/kg bodyweight of test compound in vehicle and VC groups are injected intraperitoneally or subjected to oral gavage once/twice daily for 2-4 weeks, after which blood glucose levels are measured as described above.

Alternatively, blood glucose measurements on fed Ob/Ob mice (Taconic), is performed. Mice, 6-7 week of age are distributed to a vehicle control group (VC) or a test compound treatment group, so that the mean level of blood glucose is equal between the groups. 1-30 mg/kg (e.g. 6 mg/kg) bodyweight of test compound in vehicle and VC groups are subjected to oral gavage twice daily for 18 days, after which blood glucose levels are measured as described above.

### Results

Blood glucose levels are attenuated by treatment with the test compounds.

### Method (II)

### Aim

The aim of this study was to verify the efficacy of compound of Example 1 in the diabetic ob/ob mouse with regard to correction of the metabolic disorder hyperglycemia. Ob/ob mice were gavaged twice daily with compound of Example 1 and the effect of the compound on levels of blood glucose were assessed and the results were compared to a concurrent control group gavaged with vehicle.

### Materials and Methods

### Materials—See Test C (Method (II))

### Animals

Male B6.V-Lep^{ob}/JBomTac (model number OB-M) mice were bred and delivered by Taconic. Animals were housed in Umea University animal facility in transparent polycarbonate cages, with wood chip bedding at a 12 h light/darkness cycle, a temperature of ˜21° C., and a relative humidity of ˜50% throughout the accommodation and dosing periods. 5 animals were housed in each cage with free access to standard rodent chow (CRM(E)Rodent, Special Diets Services, Scanbur BK, Sweden) and tap water. All animal experiments were approved by the Local Ethics Review Committee on Animal Experiments, Umea Region.

### Animal Experimental Procedures

In vivo potency and efficacy were determined in groups of 10 mice. Male ob/ob mice, 6 to 7 weeks of age were gavaged (5 ml/kg bodyweight) twice-daily (8:00-9:00 A.M. and 4:00-5:00 P.M.) with compound of Example 1 (30 mg/kg bodyweight) for 18 days. Blood samples were drawn from the tail vein from fed animals at day 0, 10 and at day 18 of dosing for analysis of blood glucose.

### Analytic Methods

Blood glucose levels were measured by using a Glucometer Elite (Bayer) according to the manufacturer's recommendations.

### Data Analysis

Data in the figures are presented as means±SEM. P values were calculated using the Student's t-test. Values of P<0.05 (*) were considered to be statistically significant (P<0.01 ** and P<0.001 ***). Statistical analyses were performed using Microsoft Office Excel 2003.

### Results

Blood glucose levels were attenuated in Ob/Ob mice after treatment with compound of Example 1, as shown by FIG. 4.

### Test E

### Serum Triglyceride Measurements Study in Diabetic Ob/Ob Mice.

### Method (I)

### Reagents

Serum Triglyceride Determination Kit TR0100 (sigma).

Serum Triglyceride measurements on 8-9 week old Ob/Ob mice (Taconic) fasted for 4 h/unfasted/day after 4 h fast is performed. Mice are distributed to a vehicle control group (VC) or a test compound treatment group, so that mean serum triglyceride is equal between the groups. 1-20 mg/kg bodyweight of test compound in vehicle and VC groups are injected intraperitoneally or subjected to oral gavage once/twice daily for 2-4 weeks, after which serum triglyceride levels are measured as described above.

Alternatively, plasma triglyceride measurements on fed Ob/Ob mice (Taconic), is performed. Mice, 6-7 weeks of age are distributed to a vehicle control group (VC) or a test compound treatment group, so that the mean concentration of plasma triglycerides is equal between the groups. 1-15 mg/kg (e.g. 6 mg/kg) bodyweight of test compound in vehicle and VC groups are subjected to oral gavage twice daily for 20 days, after which plasma triglyceride levels are measured as described above.

### Results

Serum triglyceride levels are attenuated by treatment with the test compounds.

### Method (II)

### Aim

The aim of this study was to verify the efficacy of compound of Example 1 in the diabetic ob/ob mouse with regard to correction of the metabolic disorder hypertriglyceridemia. Ob/ob mice were gavaged twice daily with compound of example 1 and the effect of the compound on levels of plasma triglycerides were assessed and the results were compared to a concurrent control group gavaged with vehicle.

### Materials and Methods

Materials—See Test C (Method (II))

### Animals

Male B6.V-Lep^{ob}/JUmeaTac (model number UMEA-M) mice were bred and delivered by Taconic. Animals were housed in Umea University animal facility in transparent polycarbonate cages, with wood chip bedding at a 12 h light/darkness cycle, a temperature of −21° C., and a relative humidity of −50% throughout the accommodation and dosing periods. 5 animals were housed in each cage with free access to standard rodent chow (CRM(E)Rodent, Special Diets Services, Scanbur BK, Sweden) and tap water. All animal experiments were approved by the Local Ethics Review Committee on Animal Experiments, Umea Region.

### Animal Experimental Procedures

In vivo potency and efficacy were determined in groups of 10 mice. Male ob/ob mice, 6 to 7 weeks of age were gavaged (2.5 ml/kg bodyweight) twice-daily (8:00-9:00 A.M. and 4:00-5:00 P.M.) with compound of Example 1 (15 mg/kg bodyweight) for 20 days. Blood samples were drawn from the tail vein from fed animals at day 0, 14 and at day 20 of dosing for analysis of plasma triglycerides.

### Analytic Methods

Plasma triglycerides were determined with an enzymatic colorimetric assay (TRO100, Sigma-Aldrich) according to the manufacturer's recommendations.

### Data Analysis

Data in the figures are presented as means±SEM. P values were calculated using the Student's t-test. Values of P<0.05 (*) were considered to be statistically significant (P<0.01 ** and P<0.001 ***). Statistical analyses were performed using Microsoft Office Excel 2003.

### Results

Plasma triglycerides were attenuated in Ob/Ob mice after treatment with compound of Example 1, as shown by FIG. 5.

### Test F

### Activation of AMPK

### Materials and Methods

### Test Compound

The compound of Example 1 was obtained from Isosep AB, Uppsala, Sweden. A stock solution of 10 mM was prepared by dissolving the compound in 100% DMSO.

### Cell Line and Cell Culture

Human hepatoma HepG2 cells were purchased from American Type Culture Collection (ATCC, Manassas, USA). HepG2 cells were cultured in DMEM (Gibco 212885) containing 10% fetal bovine serum (Gibco 10500-064), 100 units/ml penicillin, 100 μg/ml streptomycin (Gibco 15140-122) and 1× non essential amino acids (Gibco 11140). The cells were incubated in a humidified atmosphere of 5% CO₂ at 37° C. and passaged every 3 days by trypsinization. For experiments, HepG2 cells were incubated in complete medium with 10% fetal bovine serum in 60 or 100-mm-diameter dishes and grown to ˜70-80% confluence and subjected to assays after overnight serum depletion (16 h). After incubation in serum-free DMEM, the compound of Example 1 that was dissolved in DMSO was added to the medium. The final concentration of DMSO did not exceed 0.1%, which did not affect AMPK phosphorylation.

### Western Blot Analysis

Cells were lysed in 100 mM TRIS pH 6.8, 2% w/v Sodium dodecyl sulfate (SDS), 10 mM NaF, 10 mM β-glycerophosphate, 1 mM Na Vanadate. Protein concentration of the lysates was measured by BCA protein assay kit (Pierce #23225). 25 μg protein was loaded in each well of a 4-12% bis/tris gel for AMPK detection (Criterion precast gel Bio-Rad #345-0124) or 5% Tris-HCl gel for acetyl-CoA carboxylase (ACC) detection (Criterion Precast gel Bio-Rad #345-0002) and run according to manufacturers recommendation. Gels were blotted onto nitrocellulose filters (Hybond-C extra Amersham #RPN203E). Filters were blocked in 20 mM TRIS pH 7.5, 137 mM NaCl, 25% v/v Tween20 and 5% w/v fat free powdered milk for 30 min. Filters were incubated overnight in blocking solution with phospho-AMPKαThr172, AMPKα or phospho-ACC (Ser 79) (Cell signalling #2531, #2532 and #3661). Filters were washed in 20 mM TRIS pH 7.5, 137 mM NaCl, 25% v/v Tween20 for 3×5 min. Filters were incubated in blocking solution with secondary antibody, peroxidase-conjugated Goat anti rabbit IgG (Jackson immunoResearch #111-035-003) in room temperature for 1 h. Filters were washed as above for 3×10 min. Signal was developed with SuperSignal West Dura ECL kit (Pierce #1859024) and exposed to Hyperfilm ECL (Amersham #28906837).

### Results

The compound of Example 1 stimulates AMPK phosphorylation in cultured human HepG2 hepatocytes (FIG. 6). The phosphorylation of AMPK occurred rapidly, rising to near maximal levels within 1 h, and was sustained for 24 h (FIG. 1). Moreover, AMPK activation by the compound of Example 1 in HepG2 cells was further confirmed by enhanced phosphorylation of ACC (FIG. 7), the best characterized downstream substrate of AMPK.

These results, as depicted by FIGS. 6 and 7, indicate that compound of Example 1 stimulates AMPK phosphorylation and downstream activity.

### Test G

### Amelioration of TGFβ Dependent Collagen IV Secretion

### Methods and Materials

Cell culture and treatment:

Human primary mesangial cells (Lonza) were seeded at a density of 57 00 cells per well in a 24 well plate in DMEM (Gibco 212885) supplemented with Non essential amino acids (Gibco 11140) Penicillin/streptomycin (Gibco 15140-122) and 10% Foetal Calf Serum (FBS) (Gibco 10500-064)

After 24 h incubation, media was changed to DMEM (Gibco 212885) supplemented with Non essential amino acids (Gibco 11140) Penicillin/streptomycin (Gibco 15140-122) and 0.5% Foetal Calf Serum (FBS)(Gibco 10500-064) as the experimental media. After 24 h incubation, media was changed to experimental media containing either vehicle or β (0.5 ng/ml), compound of Example 1 (2.5 μM), or TGFβ (0.5 ng/ml) and compound of Example 1 (2.5 μM) in combination.

After 48 h of exposure the supernatant from each well was collected and subjected to ELISA analysis.

ELISA procedure:

Using Nunc Maxisorp microtiter plates, duplicate samples of conditioned cell culture medium (diluted 1:10) was used for coating (100 Owen, 18 h, +4° C.). After 3× washing in ELISA buffer (PBS (Gibco)+0.01% Triton X-100 (Sigma)) Collagen IV rabbit polyclonal primary antibody (Rockland) diluted 1:4000 in ELISA buffer was incubated at +4° C. for 24 h. After 3× washing HRP-conjugated anti-rabbit polyclonal antibody (Jackson Immunochemicals) diluted 1:10000 in ELISA buffer was incubated at room temp for 2 h. After 3× washing in ELISA buffer, reactions were visualised using 100 μl/well TMB liquid substrate system for ELISA (Sigma) After maximum 30 min reactions were stopped by addition of 25 μl 1 M H₂SO₄ and immediately analysed at 450 nm using a Multiskan EX plate reader (Thermo Labsystems).

### Results

As shown by FIG. 8, compound of Example 1 can ameliorate the TGFβ dependent collagen IV secretion in human primary mesangial cells.

1. A compound of formula I, 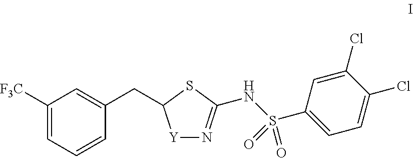
wherein:
Y represents —C(O)— or —S(O)₂—,
or a pharmaceutically-acceptable salt or solvate, or a pharmaceutically functional derivative thereof.

2. A compound as claimed in claim 1 that is: 5-(3-(trifluoromethyl)benzyl)-2-(3,4-dichlorophenyl)sulfonyl-iminothiazolidin-4-one: 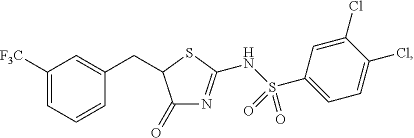
or a pharmaceutically-acceptable salt or solvate, or a pharmaceutically functional derivative thereof.

3. (canceled)

4. A pharmaceutical formulation including a compound as defined in claim 1, or a pharmaceutically-acceptable salt or solvate, or a pharmaceutically functional derivative thereof, in admixture with a pharmaceutically acceptable adjuvant, diluent or carrier.

5. A combination product comprising:
(A) the compound of formula I as defined in claim 1, or a pharmaceutically-acceptable salt or solvate, or a pharmaceutically functional derivative thereof; and
(B) another therapeutic agent useful in the treatment of a disorder or a condition caused by, linked to, or contributed to by, excess adiposity and/or hyperinsulinemia,
wherein each of components (A) and (B) is formulated in admixture with a pharmaceutically-acceptable adjuvant, diluent or carrier.

6. A combination product as claimed in claim 5 which comprises a pharmaceutical formulation including the compound of formula I, or a pharmaceutically-acceptable salt or solvate, or a pharmaceutically functional derivative thereof; the other therapeutic agent useful in the treatment of a disorder or a condition caused by, linked to, or contributed to by, excess adiposity and/or hyperinsulinemia; and the pharmaceutically-acceptable adjuvant, diluent or carrier.

7. A combination product as claimed in claim 5, which comprises a kit of parts comprising components:
(a) a pharmaceutical formulation including the compound of formula I, or a pharmaceutically-acceptable salt or solvate, or a pharmaceutically functional derivative thereof, in admixture with a pharmaceutically-acceptable adjuvant, diluent or carrier; and
(b) a pharmaceutical formulation including the other therapeutic agent useful in the treatment of a disorder or a condition caused by, linked to, or contributed to by, excess adiposity and/or hyperinsulinemia in admixture with a pharmaceutically-acceptable adjuvant, diluent or carrier,
which components (a) and (b) are each provided in a form that is suitable for administration in conjunction with the other.

8. A kit of parts as claimed in claim 7, wherein components (A) and (B) are suitable for sequential, separate and/or simultaneous use in the treatment of a disorder or a condition caused by, linked to, or contributed to by, excess adiposity and/or hyperinsulinemia.

9. A combination product as claimed in claim 5, wherein the other therapeutic agent is selected from insulin, an insulin secretagogue, metformin, a peroxisome proliferator-activated receptor agonist, an α-glucosidase inhibitor, a GLP-1 receptor agonist, a DPP-IV inhibitor, exenatide, an inhibitor of 11-beta hydroxysteroid dehydrogenase type 1, an enzyme associated with conversion of cortisone to cortisol in the liver and adipose tissue, and GLP-1 or gastric inhibitory polypeptide, or a biologically active fragment, variant, fusion or derivative of either of these peptides.

10. (canceled)

11. (canceled)

12. A method of treatment of a disorder or a condition caused by, linked to, or contributed to by, excess adiposity and/or hyperinsulinemia, which method comprises the administration of an effective amount of the compound of formula I as defined in claim 1, or a pharmaceutically-acceptable salt or solvate, or a pharmaceutically functional derivative thereof, to a patient in need of such treatment.

13. A method as claimed in claim 12, wherein the disorder or condition is hyperinsulinemia or an associated condition.

14. A method as claimed in claim 13, wherein the condition is selected from hyperinsulinemia, type 2 diabetes, glucose intolerance, insulin resistance, metabolic syndrome, dyslipidemia, hyperinsulinism in childhood, hypercholesterolemia, high blood pressure, obesity, a fatty liver condition, diabetic nephropathy, diabetic neuropathy, diabetic retinopathy, a cardiovascular disease, atherosclerosis, a cerebrovascular condition, stroke, systemic lupus erythematosus, a neurodegenerative disease, Alzheimer's disease, polycystic ovary syndrome, progressive renal disease and chronic renal failure.

15. A method as claimed in claim 14, wherein the condition is hyperinsulinemia or type 2 diabetes.

16. A kit of parts comprising:
(I) one of components (a) and (b) as defined in claim 7, together with
(II) instructions to use that component in conjunction with the other of the two components.

17. A method of making a kit of parts as defined in claim 7, which method comprises bringing component (a) into association with component (b), thus rendering the two components suitable for administration in conjunction with each other.

18. A process for the preparation of a compound of formula I as defined in claim 1, which process comprises:
(i) for compounds of formula I in which Y represents —C(O)—, reaction of either:
(A) a compound of formula II, 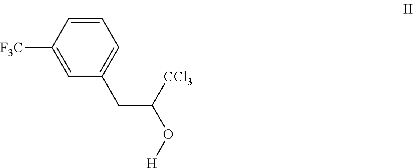
(B) a compound of formula III, 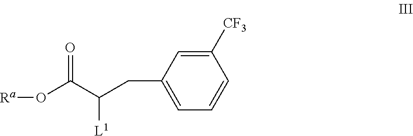
wherein R^{a} represents C₁₋₆ alkyl, L¹ represents a suitable leaving group; or
(C) a compound of formula IV, 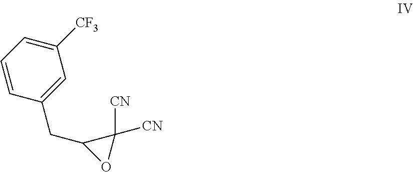
with, in each case, a compound of formula V, 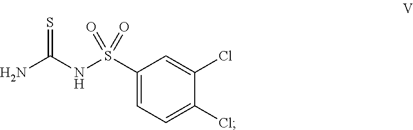
(ii) reaction of a compound of formula VI, 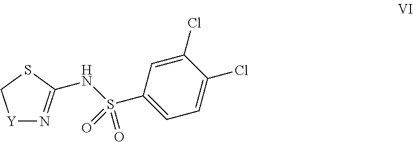
wherein Y is as defined in claim 1, with a compound of formula VII, 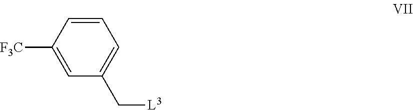
wherein L³ represents a suitable leaving group;
(iii) reaction of a compound of formula VIII, 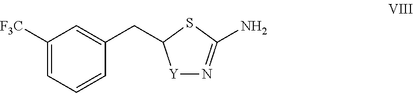
in which Y is as defined in claim 1, with a compound of formula IX, 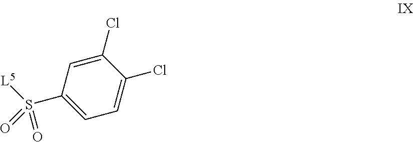
wherein L⁵ represents a suitable leaving group;
(iv) for compounds of formula I in which Y represents —SO₂—, reaction of a compound of formula I×A, 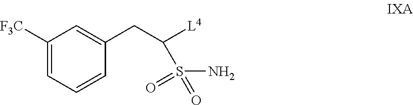
wherein L⁴ represents a suitable leaving group, with a compound of formula X 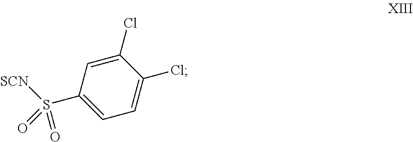
(v) reaction of a compound of formula IXB, 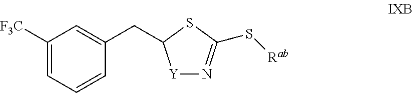
wherein R^{ab} represents C₁₋₆ alkyl (optionally substituted by one or more halo atoms), with 3,4-dichlorobenzenesulfonamide.

19. The pharmaceutical formulation of claim 4 wherein the compound is 5-(3-(trifluoromethyl)benzyl)-2-(3,4-dichlorophenyl)sulfonyl-iminothiazolidin-4-one: 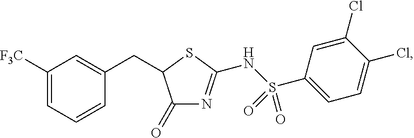
or a pharmaceutically-acceptable salt or solvate, or a pharmaceutically functional derivative thereof.

20. The method of claim 12 wherein the compound is 5-(3-(trifluoromethyl)benzyl)-2-(3,4-dichlorophenyl)sulfonyl-iminothiazolidin-4-one: 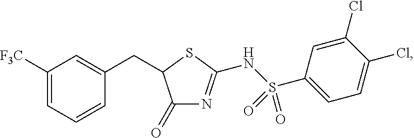
or a pharmaceutically-acceptable salt or solvate, or a pharmaceutically functional derivative thereof.

21. The method of claim 15 wherein the compound is 5-(3-(trifluoromethyl)benzyl)-2-(3,4-dichlorophenyl)sulfonyl-iminothiazolidin-4-one: 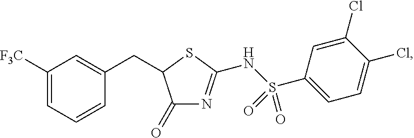
or a pharmaceutically-acceptable salt or solvate, or a pharmaceutically functional derivative thereof.
